(19) United States
(12) Patent Application Publication
(10) Pub. No.: US 2026/0295209 A1
(43) Pub. Date: Oct. 1, 2026

(54) SINGLE UNITARY PIECE BRAIDED WOVEN CONSTRUCT ENDOVASCULAR CATHETER WITH ASYMMETRIC AGGREGATE FLEXURAL STRENGTHS AMONG FLEXURAL PLANES

(71) Applicant: DEPUY SYNTHES PRODUCTS, INC., RAYNHAM, MA (US)

(72) Inventors: PEDRO D. PEDROSO, PARKLAND, FL (US); CHADWIN HANNA, JR., GAINSVILLE, FL (US)

(73) Assignee: DEPUY SYNTHES PRODUCTS, INC., RAYNHAM, MA (US)

(21) Appl. No.: 19/096,913

(22) Filed: Apr. 1, 2025

(51) Int. Cl. A61M 25/00

(52) U.S. Cl. CPC A61M 25/0012

(57) ABSTRACT

A method for producing a catheter device as a unitary single piece tubular braided woven construct has at least one spiral pattern extending in a longitudinal direction. A multi-carrier braiding system includes a support plate on which is mounted a plurality of horn gears are arranged radially in a circle. Each horn gear is rotatable about a respective central horn gear axis and has associated therewith at least one bobbin carrier configured to receive a respective bobbin of a wound wire. The plurality of horn gears represents multiple horn gear polar pairs with each horn gear polar pair of the multiple horn gear polar pairs representing two horn gears disposed radially in the circle 180 degrees of one another defining a corresponding flexural plane. The method includes creating the at least one spiral pattern by establishing asymmetric aggregate flexural strengths among the flexural planes.

### FIELD

The present disclosure relates to an access catheter for medical treatment and, in particular, to creation of a braided woven construct using a braiding machine including a plurality of horn gears collectively arranged radially in a circle, and those horn gears arranged radially 180 degrees of one another represent a horn gear polar pair defining a flexural plane the wires along that flexural plane having an associated aggregate flexural strength wherein the aggregate flexural strength among each of the plurality of flexural planes being asymmetrical, non-uniform or differential to create at least one spiral pattern (i.e., effect) in the braided woven construct in a longitudinal direction.

### BACKGROUND

Braided woven constructs for use as an access catheter are used extensively in a wide variety of medical treatments. Currently catheters are manufactured by a conventional braided woven construct including any desired number of typically 16-32 separate individual wires. Access catheters for vascular interventions are typically constructed of four general components described radially outward in series starting with the innermost component: (i) an inner liner (e.g., fluorinated polymer such as PTFE (Teflon)) defining a lumen extending in a longitudinal/axial direction and having an inner diameter; (ii) a plurality of wires (e.g., stainless steel or a polymer) having a radial cross-sectional shape (e.g., round or rectangular/flat) configured as either a braided wire design (e.g., one over one pattern; one over two pattern), stand-alone coiled wire design, or some combination of separate braided and coiled design to form a frame or skeleton; (iii) a polymer extrusion (e.g., Nylon or urethane based material); and (iv) lubricious outer coating/layer/jacket (e.g., spray/dip coated hydrophilic fluid cured onto discrete sections of the outer surface of the assembled catheter representing the outer diameter).

Focus of the present disclosure is the (ii) component (i.e., metal wires) forming the frame or skeleton of the catheter shaft providing structure and strength to the braided wire construct. The polymer extrusions are melted over the braided wire construct to form a reinforced composite of plastic and metal wires.

As with all designs there are advantages and disadvantages to using a braided wire design versus a coiled wire design, and vice versa. Capitalizing on the benefits associated with each configuration some catheters utilize a combination or hybrid of braids and coils, e.g., as two separate components, coils are placed over braids and vice versa. The hybrid braid-coil configuration (e.g., braid over coil or coil over braid) exhibits both the advantageous and disadvantageous characteristics associated with each design. One disadvantage associated with the hybrid braid-coil configuration is an undesirable increase in radial thickness of the wall of the catheter due to the presence of extra wires. Wall thickness may be mitigated by reducing the diameter of the wires, however, while sacrificing the advantages associated with each design.

It is therefore desirable to develop an improved braided configuration maintaining all the advantages associated with each design configuration (e.g., braided and coiled) without sacrificing (i.e., increasing) wall thickness.

### SUMMARY

An aspect of the present disclosure relates to an improved braided access catheter maintaining all the advantages associated with each design configuration (e.g., braided and coiled) without sacrificing (i.e., increasing) radial wall thickness by creating a unitary single piece braided woven construct having at least one spiral pattern (i.e., effect) extending in a longitudinal direction created by intentionally establishing asymmetrical aggregate flexural strengths among the plurality of flexural planes as defined by respective horn gear polar pairs radially arranged 180 degrees of one another.

While another aspect of the present disclosure is directed to an access catheter with a unitary single piece braided woven construct with at least one flexural plane as defined by a respective horn gear polar pair having a differential aggregate flexural strength by: (i) selecting for at least one wire associated therewith the at least one flexural plane a different physical characteristic (e.g., material; outer diameter; and/or radial cross-sectional shape such as round, flat/rectangular, triangular, etc.); and/or (ii) leaving empty (i.e., free of an associated bobbin) one or more of the bobbin carriers associated with the horn gear pair defining at least one flexural plane.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices of the present disclosure, by way of example only, not by way of limitation.

FIG. 1 is a radial cross-sectional view of an exemplary 16 carrier, 8 horn gear braiding system.

FIG. 2A is a radial cross-sectional view of an example 16 carrier, 8 horn gear braiding system.

FIG. 2B is a longitudinal side view of the produced unitary single piece braided woven construct using the braiding system arrangement of FIG. 2A, wherein the differential larger aggregate flexural strength of the wires along the 3^{rd} flexural plane defined through the 3^{rd} horn gear polar pair create a single spiral pattern extending in the longitudinal direction.

FIG. 2C is a cross-sectional view of the unitary single piece braided woven construct of FIG. 2B taken along line 2C-2C of FIG. 2B and looking in the direction of the arrows and shown in an isometric view.

FIG. 2D is a cross-sectional view of the unitary single piece braided woven construct of FIG. 2B taken along line 2D-2D of FIG. 2B and looking in the direction of the arrows and shown in an isometric view.

FIG. 2E is a cross-sectional view of the unitary single piece braided woven construct of FIG. 2B taken along line 2E-2E of FIG. 2B and looking in the direction of the arrows and shown in an isometric view.

FIG. 2F is a cross-sectional view of the unitary single piece braided woven construct of FIG. 2B taken along line 2F-2F of FIG. 2B and looking in the direction of the arrows and shown in an isometric view.

FIG. 3A is a radial cross-sectional view of an example 16 carrier, 8 horn gear braiding system.

FIG. 3B is a longitudinal side view of the produced unitary single piece braided woven construct using the braiding system arrangement of FIG. 3A, wherein the two flexural planes equal in aggregate flexural strength (e.g., 1^{st} flexural plane defined through the 1^{st} horn gear polar pair; and 4^{th} flexural plane defined through the 4^{th} horn gear polar pair) create a first spiral pattern extending in the longitudinal direction, while the aggregate flexural strength along the other two other flexural planes (e.g., 2^{nd} flexural plane defined through the 2^{nd} horn gear polar pair; and 3^{rd} flexural plane defined through the 3^{rd} horn gear polar pair) create a second spiral pattern extending in the longitudinal direction.

FIG. 4A is a radial cross-sectional view of an example 16 carrier, 8 horn gear braiding system.

FIG. 4B is a longitudinal side view of the produced unitary single piece braided woven construct using the braiding system arrangement of FIG. 4A depicting four separate spiral patterns extending in the longitudinal direction created by the different aggregate flexural strengths along each of the four flexural planes.

FIG. 5A is a radial cross-sectional view of an example 16 carrier, 8 horn gear braiding system.

FIG. 5B is a longitudinal side view of the produced unitary single piece braided woven construct using the braiding system arrangement of FIG. 5A depicting four separate spiral patterns extending in the longitudinal direction created by the different aggregate flexural strengths along each of the four flexural planes.

FIG. 6A is a radial cross-sectional view of an example 16 carrier, 8 horn gear braiding system.

FIG. 6B is a longitudinal side view of the produced unitary single piece braided woven construct using the braiding system arrangement of FIG. 2A, wherein the two flexural planes equal in aggregate flexural strength (e.g., 1^{st} flexural plane defined through the 1^{st} horn gear polar pair; and 3^{rd} flexural plane defined through the 3^{rd} horn gear polar pair) create a first spiral pattern extending in the longitudinal direction, while the differential aggregate flexural strength along each of the other two other flexural planes (e.g., 2^{nd} flexural plane defined through the 2^{nd} horn gear polar pair; and 4^{th} flexural plane defined through the 4^{th} horn gear polar pair) create a second and third, respective, spiral pattern extending in the longitudinal direction.

FIGS. 7A and 7B are a table of the collective data set associated with each of the example braiding machine arrangements in FIGS. 2A, 3A, 4A, 5A & 6A.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

As used herein, the terms “inner surface” and “outer surface” refer to the collective radial arrangement in a circle of the plurality of horn gears of the braiding machine defining a collective radial inner surface and a collective radial outer surface. Applying these terms to the examples illustrated herein, the 8 horn gears are collectively radially arranged in a circle each horn gear has two associated bobbin carriers, e.g., an inner bobbin carrier disposed on the collective radial inner surface of the plurality of horn gears collectively radially arranged in the circle and an outer bobbin carrier disposed on the collective radial outer surface of the plurality of horn gears collectively radially arranged in the circle. Each bobbin carrier (e.g., inner bobbin carrier and/or outer bobbin carrier) is potentially fitted with at least one corresponding bobbin (i.e., spool) of wound wire. It is possible for any one or more bobbin carriers (e.g., inner bobbin carrier and/or outer bobbin carrier) to be empty (i.e., free or devoid of an associated bobbin of wound wire).

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

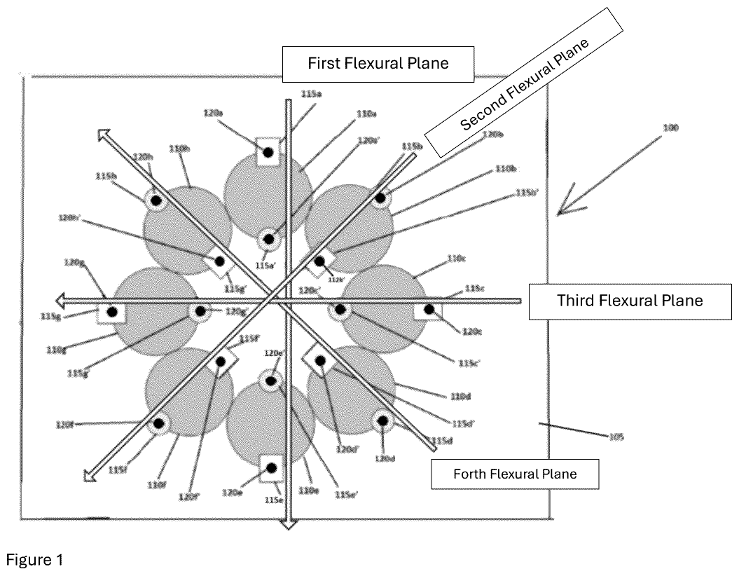

The present disclosure is directed to a method for creating a tubular braided construct for medical use (e.g., access catheter). Braiding machines are used to manufacture tubular braided woven constructs for medical use such as, for example, catheters or stents. FIG. 1 is a radial cross-sectional view of an exemplary 16 carrier, 8 horn gear braiding system illustrating the 4 horn gear polar pairs (each pair representing two horn gears arranged 180 degrees radially of each other) (e.g., 1^{st} horn gear polar pair-horn gears 110a & 110e; 2^{nd} horn gear polar pair-horn gears 110b & 110f; 3^{rd} horn gear polar pair-horn gears 110c & 110g; 4^{th} horn gear polar pair-horn gears 110d & 110h) and corresponding 4 flexural planes (e.g., 1^{st} flexural plane A defined through the 1^{st} horn gear polar pair; 2^{nd} flexural plane B defined through the 2^{nd} horn gear polar pair; 3^{rd} flexural plane C defined through 3^{rd} horn gear polar pair; 4^{th} flexural plane D defined through 4^{th} horn gear polar pair). For the sake of clarity in the drawings, each flexural plane A, B, C, D is represented as being offset from the center as shown with a dotted line in the construct of FIG. 1. But in practice, the flexural planes intersect the centers of all crossing wires.

Referring to the example in FIG. 1, the braiding machine includes a plate 105 and a gear train comprising a plurality of rotating horn gears 110a-110 h collectively radially arranged in a Maypole type braiding configuration. FIG. 1 is an example 16 carrier tubular braiding machine or system including 8 rotating horn gears 110a-110h collectively radially arranged in a circle defining a collectively radially arranged inner surface and a collectively radially arranged outer surface. Each horn gear 110a-110h has associated therewith two bobbin carriers, e.g., an outer bobbin carrier disposed on the collectively radially arranged outer surface 115a-115h and an inner bobbin carrier disposed on the collectively arranged inner surface 115a′-115h′. Each bobbin carrier (e.g., inner bobbin carrier and/or outer bobbin carrier) of any of the plurality of the horn gears 110l-110h may be configured to receive (i.e., be fitted with) one or more than one bobbin (i.e., spool) 120a-120h, 120a′-120h′ of wound wire. Any bobbin carrier potentially may be fitted or receive a replaceable bobbin of wound wire (e.g., metal or polymer) to be woven by the braiding machine. In the example arrangement of FIG. 1, each outer bobbin carrier 115a-115h is configured to receive a single bobbin 120a-120h, while each inner bobbin carrier 115a′-115h′ is configured to receive a single bobbin 120a′-120h′. It is also possible that a particular bobbin carrier (e.g., inner bobbin carrier and/or outer bobbin carrier) of one of the plurality of horn gears 110a-110h may remain empty, free, devoid or without an associated bobbin. Movement of the horn gears 110a-110h within the tracks or grooves defined in the plate 105 creates the tubular braided woven construct in a desired pattern or configuration. The produced braided construct would come out perpendicularly (z axis) from the two-dimensional plane (x-y plane) represented in FIG. 1. Each bobbin carrier 115a-115h, 115a′-115h′ has a particular shape (e.g., round or square), as desired, and need not all be the same.

The exemplary braiding machine depicted throughout the drawings has 8 horn gears 110a-110h collectively radially arranged in a circle representing 4 horn gear polar pairs, wherein the term “horn gear polar pair” is herein defined as two horn gears disposed radially 180 degrees of one another. Each horn gear polar pair together defines what is herein referred to as a “flexural plane” or “bending plane.” Specifically, the example 8 horn gears represent 4 horn gear polar pairs (e.g., 1^{st} horn gear polar pair includes horn gears 110a & 110e; 2^{nd} horn gear polar pair includes horn gears 110b & 110f; 3^{rd} horn gear polar pair includes horn gears 110c & 110g; 4^{th} horn gear polar pair includes horn gears 110d & 110h). These 4 horn gear polar pairs represent respective 4 flexural planes, i.e., one flexural plane defined by each horn gear polar pair. As represented by the four arrows, the 4 flexural planes in FIG. 1 include: 1^{st} flexural plane-defined through the 1^{st} horn gear polar pair including horn gears 110a & 110e; 2^{nd} flexural plane-defined through the 2^{nd} horn gear polar pair including horn gears 110b & 110f; 3^{rd} flexural plane-defined through the 3^{rd} polar pair of horn gears 110c & 110g; and 4^{th} flexural plane defined through the 4^{th} horn gear polar pair including horn gears 110d & 110h). If the braiding machine or system has an odd number of horn gears one horn gear will neither have a counterpart horn gear disposed radially 180 degrees thereof with which to form a polar pair nor define an associated flexural plane.

By varying the aggregate flexural strength or (i.e., stiffness) of the wires or filaments along at least one of the respective flexural planes a unique and specifically tailored tubular braided woven construct is formed as a unitary single piece with at least one spiral pattern extending in a longitudinal direction of wires associated with each flexural plane having a differential aggregate flexural strength. Varying the physical characteristics or properties of specific wires associated with horn gears in a particular flexural plane produces a different aggregate flexural strength (i.e., stiffness) in comparison to the aggregate flexural strength in other flexural planes thereby creating one or more spiral patterns extending in the longitudinal direction down the length of the braided woven construct. Those wires associated with the flexural plane having a different aggregate flexural strength (i.e., stiffness) produce an integral spiral pattern (resembling that of a helix) compared to the other wires extending down the created braided woven construct in the longitudinal/axial direction. Such integral spiral pattern is different than merely wrapping a separate standalone coil component around a formed braided woven construct of wires all identical in physical characteristics exhibiting symmetric, uniform or identical flexural strength among the various flexural planes. Rather, the at least one spiral pattern created in accordance with the present disclosure is attributable to the aggregate flexural strength (i.e., stiffness) among the defined plurality of flexural planes being asymmetrical or non-uniform realized by: (i) differing the physical characteristic associated with one or more wires; or (ii) leaving one or more of the bobbin carriers empty (i.e., free of an associated bobbin). Thus, by selecting the physical characteristics of the wires a specific differential aggregate flexural strength among the plurality of flexural planes is realized in the produced braided woven construct to create one or more distinct spiral patterns.

This braided woven construct of asymmetric aggregate flexural re (i.e., differential stiffness) among the flexural planes in accordance with the present disclosure behaves like a coil by exhibiting relative “bounce.” However, the braided woven construct of asymmetric aggregate flexural strength among the flexural planes in accordance with the present disclosure also prevents the axial tube from stretching in a longitudinal/axial direction, not achieved by a conventional standalone coil. Accordingly, catheters in accordance with the present disclosure having a braided woven construct of asymmetric aggregate flexural strength among the flexural planes exhibit enhanced flexibility in contrast to a braided woven structure of uniform or symmetric aggregate flexural strength equal (i.e., the same) among the flexural planes thereby behaving didactically. That is, when encountering a bend or curve in the vasculature the catheter created using the braided woven construct of asymmetric aggregate flexural strength among the flexural planes in accordance with the present disclosure recoils or bounces as forward (i.e., in the distal direction) compressive forces continue to be applied on the catheter by the interventionalist or physician.

Such recognition of differential aggregate flexural strength (i.e., differential stiffness) among the plurality of flexural planes is based on the following principles.

Based on beam deflection theory and mechanics of materials the deflection (d) of a beam configured in a symmetric three-point (x, y, z) bend configuration (with a single point load F) is as follows:

$$d = F {L}^{3} / 48 E I ,$$

wherein
   d is the deflection of the beam,
   F is the load
   L is the length (i.e., span) of the beam
   E is the Young's modulus of elasticity (i.e., stiffness) of the beams material
   I is the moment of inertia (i.e., second moment of area) of the beam (this parameter is related to the beams cross-sectional shape).

For a round beam there are two unique radial components moments of inertia (i.e., one moment of inertia is identical in the x and y axes (Ix, Iy)

$$Iy = Ix = {piD}^{4} / 64$$

where D is the outer diameter of the beam.

While the axial (i.e., polar) component Iz in the z-direction is

$$Iz = {piD}^{4} / 32$$

where D is the outer diameter of the beam.

Combining these formulas and solving for the deflection or flexural force (i.e., flexural strength) (F) yields the following formula in the respective y and z axes (Fy, Fz):

$$Fy = 0.75 {piED}^{4} d / {L}^{3} and Fz = 1.5 {piED}^{4} d / {L}^{3}$$

Based on these equations a first beam that is more flexible will have a flexural strength (F1) (i.e., stiffness) that is less than a more rigid second beam having a flexural strength (F2), where F1<F2, (i.e., F1 is more flexible than F2).

Ignoring the complex interaction of friction within a tubular braided woven construct, for simplicity, an “aggregate flexural strength” is calculated for the braided woven construct along each unique flexural plane (i.e., bending plane), where each flexural plane (i.e., bending plane) represents two horn gears arranged radially 180 degrees of each other. In the example of FIG. 1 in each flexural plane defined by a single horn gear polar pair there are four wires that can be bent (e.g., two wires associated with each horn gear). For example, in the 1^{st} flexural plane as defined by 1^{st} horn gear polar pair there are 2 wires one wire from each of outer bobbin 120a and inner bobbin 120a′ associated with horn gear 110a and 2 wires one wire from each of outer bobbin 120e and inner bobbin 120e′ associated with horn gear 110e.

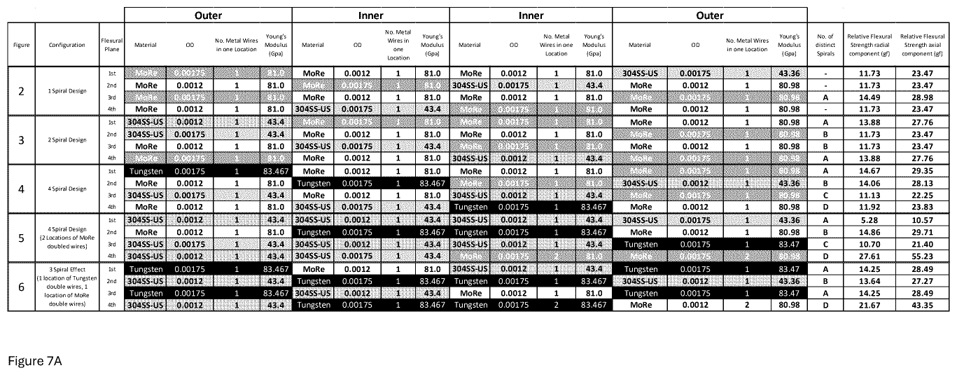

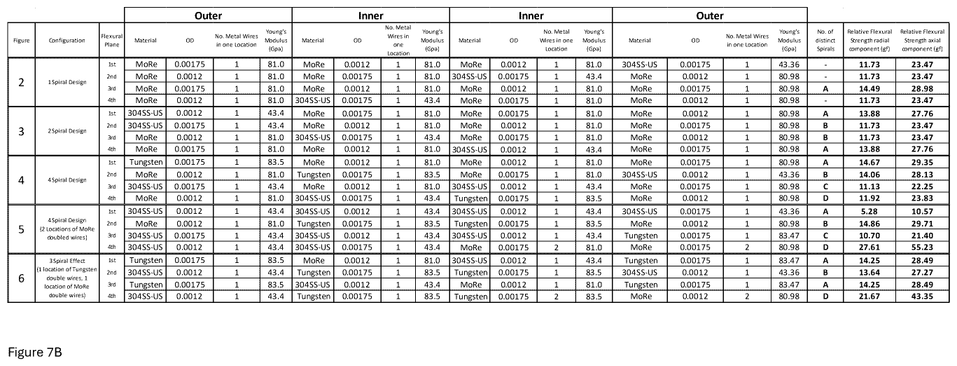

Aggregate (i.e., adding) the flexural strength axial component in the y-axis (Fy's) or flexural strength radial component in the z-axis (Fz's) for each of the four wires in a unique flexural plane represents the aggregate flexural strength (i.e., flexural force) (last two columns on the right hand side of the table in FIGS. 7A and 7B) for that particular flexural plane. Those wires (e.g., 4 wires) within a particular flexural plane (e.g., 1^{st} flexural plane) will more readily bend and be more flexible than wires (e.g., 4 wires) in another plane (e.g., 2^{nd} flexural plane) that have an aggregate flexural strength (Fy's or Fz's) larger than the latter.

In accordance with the present disclosure, it is now recognized that by varying at least one physical characteristic (e.g., material, outer diameter and/or radial cross-sectional shape) of one wire within a particular flexural plane an asymmetrical, non-uniform or differential flexural strength (i.e., differential stiffness) is achieved among the flexural planes. That is, the flexural strength along at least one desired flexural plane is intentionally differentiated from the flexural strength among the remaining flexural planes by selecting at least one wire along that at least one desired flexural plane to differentiate in at least one of the following physical characteristics, properties or parameters: (i) material; (ii) outer diameter; and/or (iii) radial cross-sectional shape. It is also possible to intentionally differentiate the flexural strength along at least one desired flexural plane by leaving empty at least one of the associated bobbin carriers (i.e., not fitted with an associated bobbin or spool) associated with the horn gear polar pair defining that particular flexural plane. A minimum flexural strength for an associated flexural plane is established by leaving empty at least one bobbin carrier associated therewith. Therefore, the intentionally differentiated flexural strength along at least one desired flexural plane may be realized by selecting: (i) one or more different physical characteristics of at least one of the wires associated therewith; and/or (ii) leaving empty at least one of the bobbin carriers associated therewith.

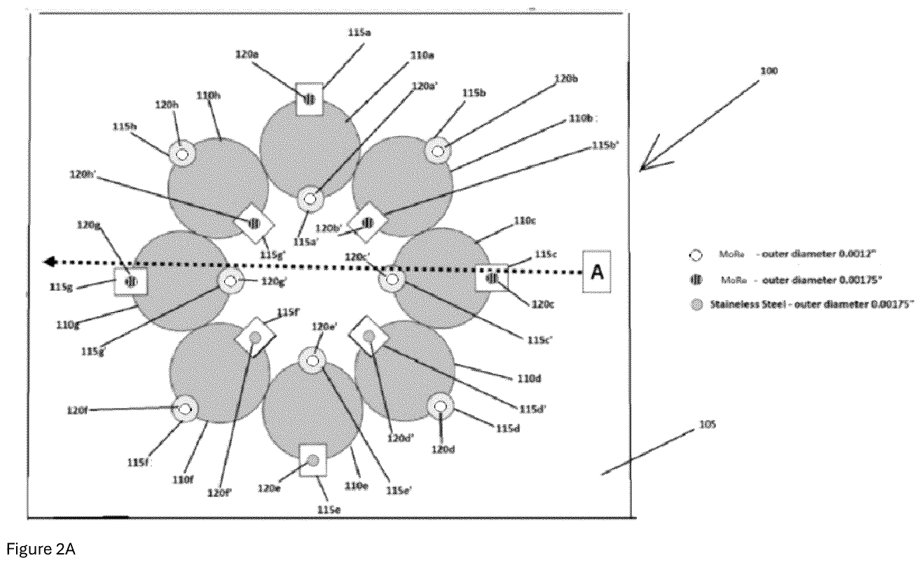

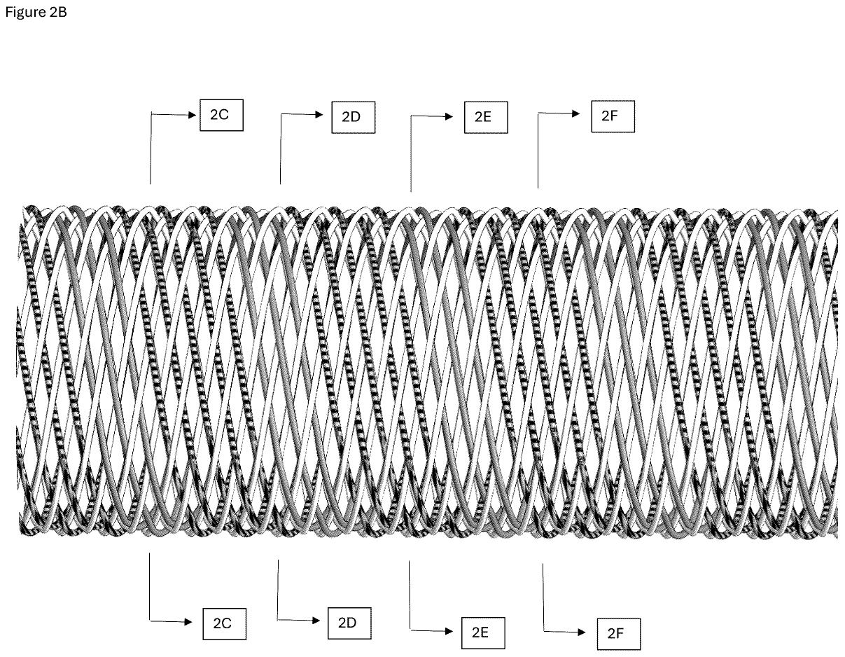

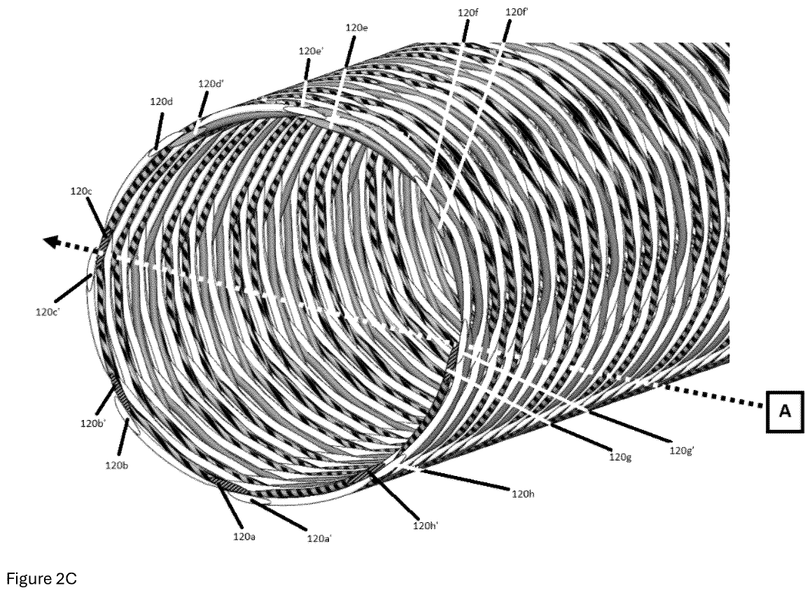

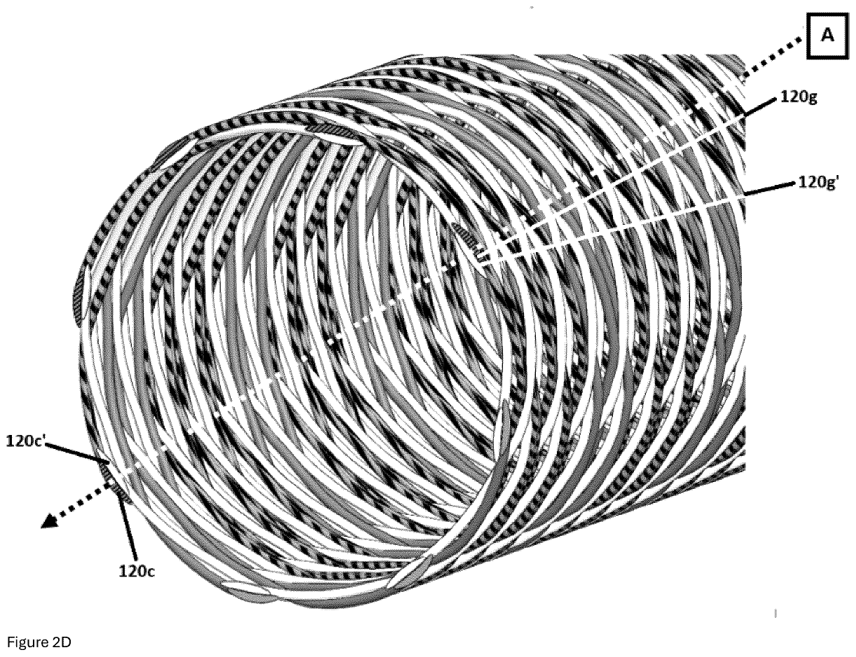

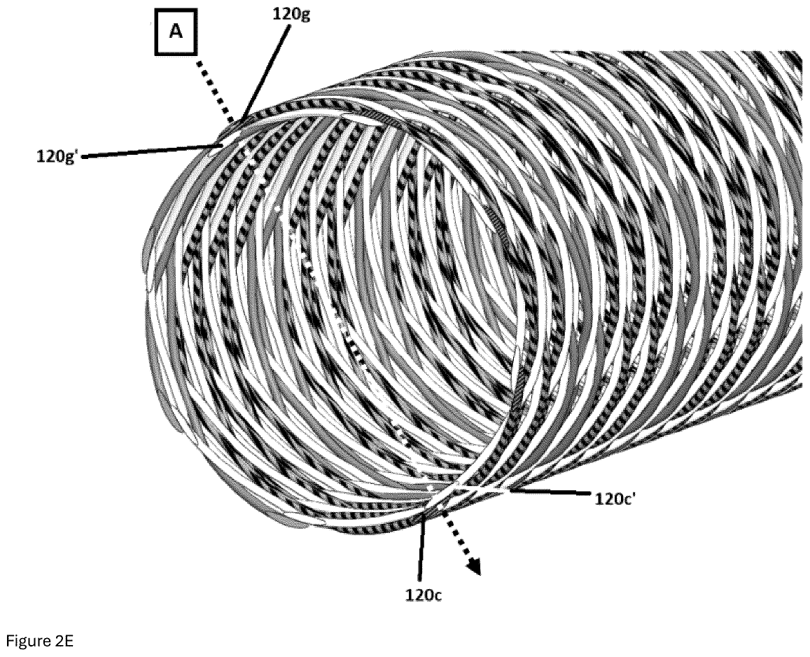

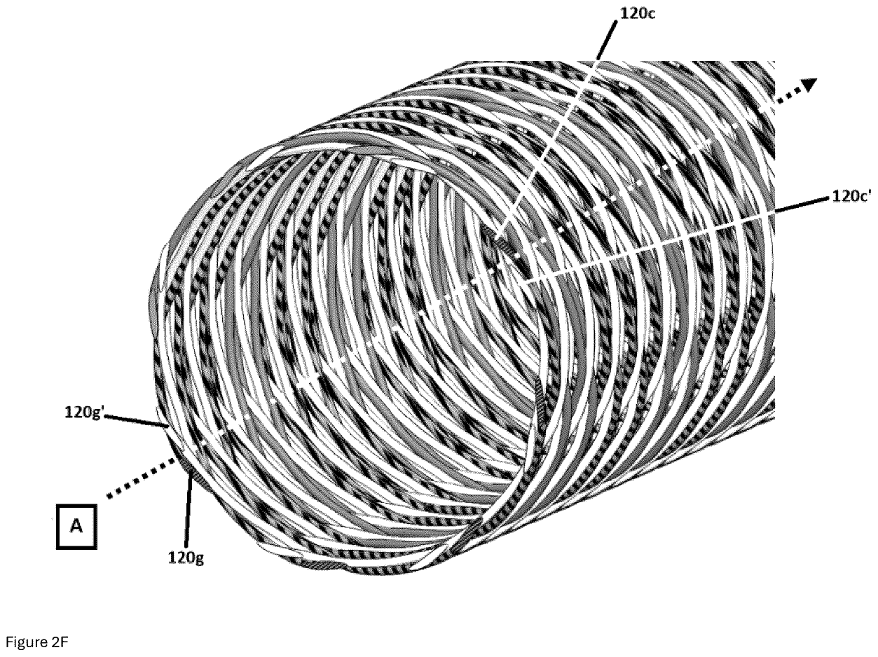

Several, non-limiting example braiding machine arrangements, are illustrated and described herein in which the flexural strength among the various flexural planes as defined by the polar pairs of horn gears are asymmetric (i.e., non-uniform, not identical, not equal or not the same). The table in FIGS. 7A and 7B (collectively referred to as FIG. 7) provides the collective data set for each example arrangement illustrated and described below. FIGS. 7A and 7B are identical except for the background hatching in FIG. 7A which corresponds to the cross hatch of the respective wires shown in FIGS. 2A-6B. For example, in FIG. 2A, in the 3^{rd} flexural plane defined through the 3^{rd} horn gear polar pair including horn gears 110c, 110g, outer bobbin wire 120c, inner bobbin wire 120c′, inner bobbin wire 120g′, and outer bobbin wire 120g are illustrated with a stripe cross-hatch, a solid white, solid white, and stripe cross-hatch, respectively. Accordingly, in FIG. 7A, The row for FIG. 2, 1 spiral design, in the 3^{rd} flexural plane row, the background hatching is striped for the two outer bobbin wires, and white for the two inner bobbin wires. The remaining illustrated rows follow the same pattern. Referring to the table in FIG. 7, the example arrangement in the first column of the table identifies the figure in the drawings. The second column describes the number of distinct spiral patterns (i.e., effects) created in the braided woven construct exhibiting an asymmetrical, non-uniform, differential flexural strength among the plurality of flexural planes for each example arrangement. Third column in the table of FIG. 7 identifies the four unique flexural planes (e.g., 1^{st} flexural plane through 4^{th} flexural plane) for each of the 8 horn gear, 16 carrier example arrangements. Specifically, 1^{st} flexural plane-defined through the 1^{st} horn gear polar pair including horn gears 110a & 110e; 2^{nd} flexural plane-defined through the 2^{nd} horn gear polar pair including horn gears 110b & 110f; 3^{rd} flexural plane-defined through the 3^{rd} polar pair of horn gears 110c & 110g; and 4^{th} flexural plane-defined through the 4^{th} horn gear polar pair including horn gears 110d & 110h.

As identified in the upper most heading (e.g., “outer”-“inner”-“outer”-“inner”) the next four columns of the table in FIG. 7 represent the data set of the four wires associated with the bobbin carriers (outer and inner) for each horn gear in a particular horn gear polar pair defining that associated flexural plane. Since the horn gears are identified alphabetically by reference element numbers 110a-110h, the first grouping of “outer” and “inner” columns (to the left) in the table of FIG. 7 describe the data set of the wire of a bobbin fitted in the outer bobbin carrier and inner bobbin carrier, respectively, associated with the horn gear identified by the lower letter alphabetically (i.e., “<alphabetically”) reference element subscript letter, while the second grouping “outer” and “inner” columns in the table describe the data set of the wire of a bobbin fitted in the outer bobbin carrier and inner bobbin carrier, respectively, of the horn gear identified by the higher letter alphabetically (i.e., “>alphabetically”) reference element subscript letter. As a subheading below the upper most heading, for each of these four wires associated with a particular horn gear polar pair, the table identifies the material, outer diameter, number of wires (e.g., 1 wire or 2 (“pair”) of wires) and Young's Modulus of Elasticity of the wire. Proceeding to the right, the next column heading “No. Spiral Effect” identifies the number of distinct spiral patterns (i.e. “effects”) created by the example arrangement, wherein each spiral effect is identified alphabetically by letter (“A”, “B”, “C” or “D”). The final two column headings “Aggregate Flexural Strength radial component (Fz)” and “Aggregate Flexural Strength axial component (Fy)” represent the aggregate or cumulative (Fz's) or (Fy's) for all four wires within the flexural plane.

FIG. 2A is a radial cross-sectional view of an example 16 carrier, 8 horn gear braiding system for a first example arrangement in which the aggregate flexural strength is equal among three flexural planes (e.g., 1^{st} flexural plane defined through the 1^{st} horn gear polar pair; 2^{nd} flexural plane defined through the 2^{nd} horn gear polar pair; and 4^{th} flexural plane defined through the 4^{th} horn gear polar pair), while in comparison thereto the differential aggregate flexural strength is larger along the 3^{rd} flexural plane defined through the 3^{rd} horn gear polar pair.

A first example is shown in FIG. 2A depicting the 8 horn gear, 16 carrier bobbin arrangement in which some wires made of Metal #1 differ in outer diameter (e.g., 0.0012″ or “0.00175”) while other wires made of Stainless Steel have the same outer diameter (e.g., 00175″). The characteristics of the 16 wires for the example arrangement in FIG. 2A are presented in the table of FIG. 7. Specifically, in the 1^{st} flexural plane defined through the 1^{st} horn gear polar pair including horn gears 110a, 110e: outer bobbin 120a is a wound wire made of Metal #1 having an outer diameter 0.00175″; inner bobbin 120a′ is a wound wire made of Metal #1 having an outer diameter 0.0012″; inner bobbin 120e′ is a wound wire made of Metal #1 having an outer diameter 0.0012″; and outer bobbin 120e is a wound wire made of Stainless Steel having an outer diameter 0.00175″. In the 2^{nd} flexural plane defined through the 2^{nd} horn gear polar pair including horn gears 110b, 110f: outer bobbin 120b is a wound wire made of Metal #1 having an outer diameter 0.0012″; inner bobbin 120b′ is a wound wire made of Metal #1 having an outer diameter 0.00175″; inner bobbin 120f′ is a wound wire made of Stainless Steel having an outer diameter 0.00175″; and outer bobbin 120f is a wound wire made of Metal #1 having an outer diameter 0.0012″. While in the 3^{rd} flexural plane defined through the 3^{rd} horn gear polar pair including horn gears 110c, 110g: outer bobbin 120c is a wound wire made of Metal #1 having an outer diameter 0.00175″; inner bobbin 120c′ is a wound wire made of Metal #1 having an outer diameter 0.0012″; inner bobbin 120g′ is a wound wire made of Metal #1 having an outer diameter 0.0012″; and outer bobbin 120g is a wound wire made of Metal #1 having an outer diameter 0.00175″. Along the 4^{th} flexural plane defined through the 4^{th} horn gear polar pair including horn gears 110d, 110h: outer bobbin 120d is a wound wire made of Metal #1 having an outer diameter 0.0012″; inner bobbin 120d′ is a wound wire made of Stainless Steel having an outer diameter 0.00175″; inner bobbin 120h′ is a wound wire made of Metal #1 having an outer diameter 0.00175″; and outer bobbin 120h is a wound wire made of Metal #1 having an outer diameter 0.0012″.

In this example arrangement of FIG. 2A, those wires having an outer diameter of 0.0012″ (within circular locations of the horn gear) do not cross one another, similarly those wires having an outer diameter of 0.00175″ (within rectangular locations of the horn gear) also do not cross each other. Instead, crossing occurs only with wires differing in outer diameter in this example (e.g., crossing of a wire having an outer diameter of 0.0012″ with a wire having an outer diameter of 0.00175″). In general wires located in the circular carrier locations within the horn gear cannot cross other wires that are within other circular locations. The same is true for wires that reside in rectangular carrier locations. In other words, wires located in the rectangular carrier locations within the horn gear do not and cannot cross other wires that are within other rectangular locations As noted in the last two columns on the right in the table of FIG. 7, the aggregate flexural strength (i.e., the radial component (Fz) or the axial component (Fy)) is equal along each of the 1^{st}, 2^{nd} & 4^{th} flexural planes, while the aggregate flexural strength (i.e., the radial component (Fz) or the axial component (Fy) along the 3^{rd} flexural plane is different. That is, the higher flexural strength along the 3^{rd} flexural plane provides greater stiffness, more rigidity, less flexibility than the lower flexural strength along the 1^{st}, 2^{nd} & 4^{th} flexural planes that are less stiff, less rigid and more flexible in comparison.

FIG. 2B is a side view of the braided woven construct produced with the braiding machine arrangement of FIG. 2A creating a single spiral pattern extending in the longitudinal/axial direction. The single spiral pattern (e.g., identified in the third column from the right in the table of FIG. 7 as coil “A”) represents those wires associated with the 3^{rd} flexural plane, while the other wires associated with the 1^{st}, 2^{nd} & 4^{th} flexural planes are represented by the surrounding wires.

Flexural plane A helically spirals around the braided woven construct. To illustrate this, flexural plane A is shown at various axial locations along the construct by dashed line A in FIGS. 2D, 2E, 2F and 2G. As shown in FIGS. 2D-2G, flexural plane A is located at various angles depending upon the cross-section because the flexural plane helically winds about the construct as one moves axially along the construct.

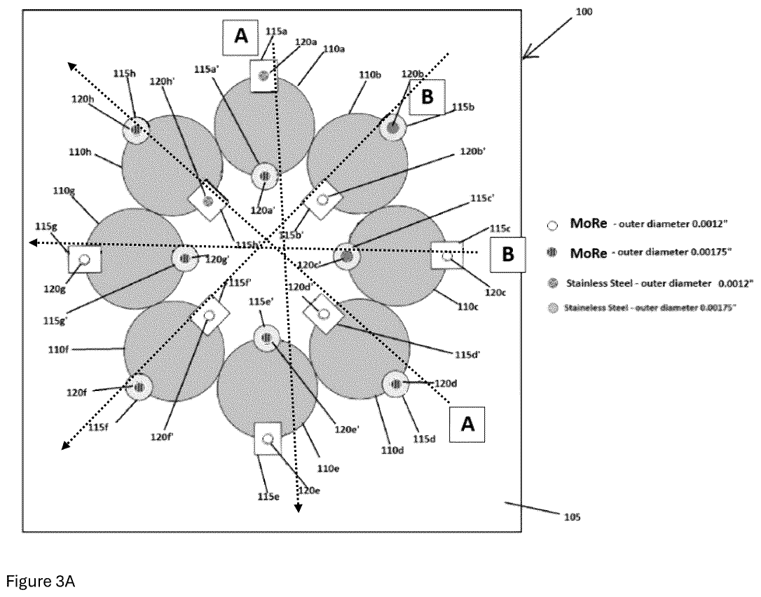

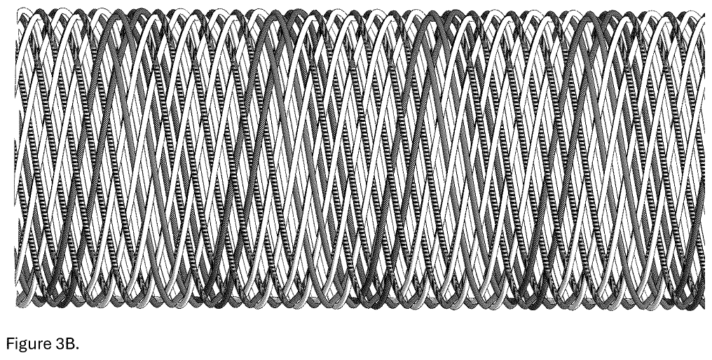

Another example 8 horn gear, 16 carrier arrangement is shown in FIG. 3A with the data set of the respective wires for this example arrangement set forth in the table of FIG. 7. Specifically, in the 1^{st} flexural plane defined through the 1^{st} horn gear polar pair including horn gears 110a, 110e: outer bobbin 120a is a wound wire made of Stainless Steel having an outer diameter 0.0012″; inner bobbin 120a′ is a wound wire made of Metal #1 having an outer diameter 0.00175″; inner bobbin 120e′ is a wound wire made of Metal #1 having an outer diameter 0.00175″; and outer bobbin 120e is a wound wire made of Metal #1 having an outer diameter 0.0012″. In the 2^{nd} flexural plane defined through the 2^{nd} horn gear polar pair including horn gears 110b, 110f: outer bobbin 120b is a wound wire made of Stainless Steel having an outer diameter 0.00175″; inner bobbin 120b′ is a wound wire made of Metal #1 having an outer diameter 0.0012″; inner bobbin 120f′ is a wound wire made of Metal #1 having an outer diameter 0.0012″; and outer bobbin 120f is a wound wire made of Metal #1 having an outer diameter 0.00175″. While in the 3^{rd} flexural plane defined through the 3^{rd} horn gear polar pair including horn gears 110c, 110g: outer bobbin 120c is a wound wire made of Metal #1 having an outer diameter 0.0012″; inner bobbin 120c′ is a wound wire made of Stainless Steel having an outer diameter 0.00175″; inner bobbin 120g′ is a wound wire made of Metal #1 having an outer diameter 0.00175″; and outer bobbin 120g is a wound wire made of Metal #1 having an outer diameter 0.0012″. Along the 4^{th} flexural plane through the 4^{th} horn gear polar pair including horn gears 110d, 110h: outer bobbin 120d is a wound wire made of Metal #1 having an outer diameter 0.00175″; inner bobbin 120d′ is a wound wire made of Metal #1 having an outer diameter 0.0012″; inner bobbin 120h′ is a wound wire made of Stainless Steel having an outer diameter 0.0012″; and outer bobbin 120h is a wound wire made of Metal #1 having an outer diameter 0.00175″.

FIG. 3A is a radial cross-sectional view of an example 16 carrier, 8 horn gear braiding system for a second example arrangement in which two flexural planes (e.g., 1^{st} flexural plane defined through the 1^{st} horn gear polar pair; and 4^{th} flexural plane defined through the 4^{th} horn gear polar pair) are equal in aggregate flexural strength but larger in comparison to the aggregate flexural strength along the two other flexural planes (e.g., 2^{nd} flexural plane defined through the 2^{nd} horn gear polar pair; and 3^{rd} flexural plane defined through the 3^{rd} horn gear polar pair).

In this example of FIG. 3A, those wires having an outer diameter of 0.0012″ do not cross one another, similarly those wires having an outer diameter of 0.00175″ also do not cross each other. Instead, crossing occurs only with wires differing in outer diameter (e.g., crossing of a wire having an outer diameter of 0.0012″ with a wire having an outer diameter of 0.00175″). As noted in the last two columns on the right in the table of FIG. 7, the aggregate flexural strength (i.e., the radial component (Fz) or the axial component r (Fy)) is equal along the 1^{st} & 4^{th} flexural planes, while the aggregate flexural strength (i.e., the radial component (Fz) or the axial component (Fy)) along the 2^{nd} & 3^{rd} flexural plane is also equal, but different from that of the 1^{st} & 4^{th} flexural planes. That is, the higher aggregate flexural strength (i.e., stiffer, more rigid, less flexibility) along the 1^{st} & 4^{th} flexural planes compared to the lower aggregate flexural strength (i.e., less stiff, less rigid, greater flexibility) along the 2^{nd} & 3^{rd} flexural planes.

FIG. 3B is a longitudinal side view of the produced unitary single piece braided woven construct using the braiding system arrangement of FIG. 3A, wherein the two flexural planes equal in aggregate flexural strength (e.g., 1^{st} flexural plane defined through the 1^{st} horn gear polar pair; and 4^{th} flexural plane defined through the 4^{th} horn gear polar pair) create a first spiral pattern extending in the longitudinal direction, while the aggregate flexural strength along the other two other flexural planes (e.g., 2^{nd} flexural plane defined through the 2^{nd} horn gear polar pair; and 3^{rd} flexural plane defined through the 3^{rd} horn gear polar pair) create a second spiral pattern extending in the longitudinal direction.

FIG. 3B is a side view of the braided woven construct produced with the arrangement of FIG. 3A creating two distinct spiral patterns extending in the longitudinal/axial direction. One of the spiral patterns represent those wires associated with the 1^{st} & 4^{th} flexural planes, respectively, having a higher aggregate flexural strength (e.g., coil “A”), while the other spiral pattern represents those wires associated with the 2^{nd} & 3^{rd} flexural planes, respectively, of a lower aggregate flexural strength (e.g., coil “B”).

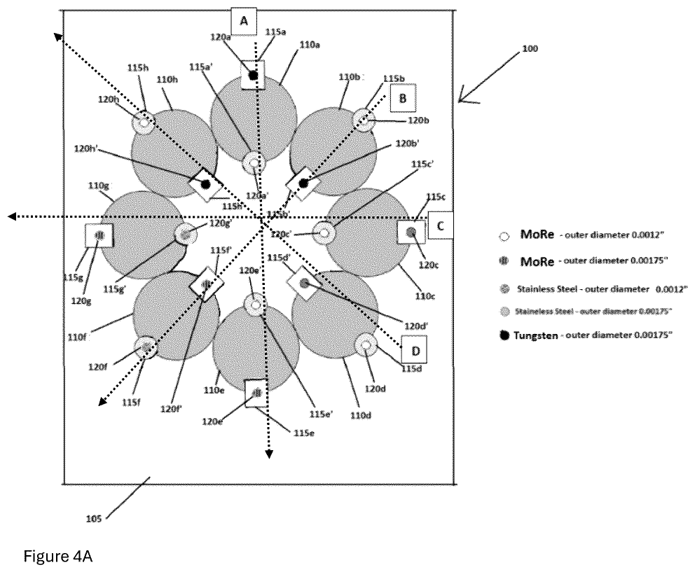

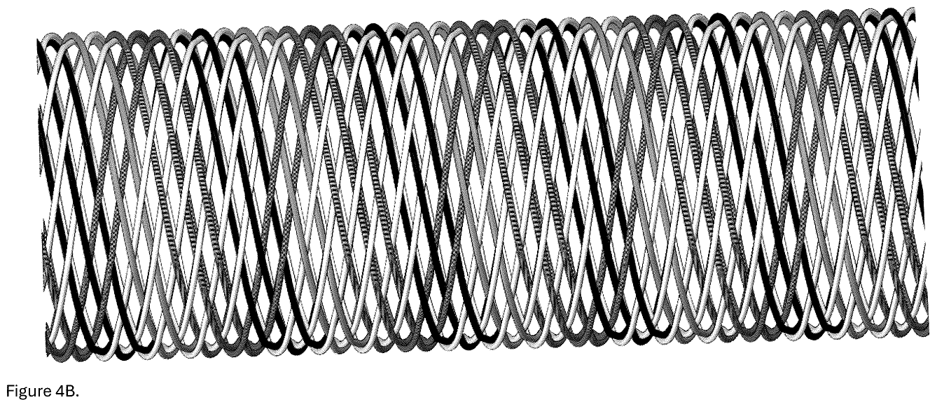

FIG. 4A is a radial cross-sectional view of an example 16 carrier, 8 horn gear braiding system for a third example arrangement in which the aggregate flexural strength differs in each of the four flexural planes (e.g., 1^{st} flexural plane defined through the 1^{st} horn gear polar pair; 2^{nd} flexural plane defined through the 2^{nd} horn gear polar pair; 3^{rd} flexural plane defined through the 3^{rd} horn gear polar pair; and 4^{th} flexural plane defined through the 4^{th} horn gear polar pair);

In the FIG. 4A example, the characteristics of the wires for the example arrangement in FIG. 4A are presented in the table of FIG. 7. Specifically, in the 1^{st} flexural plane defined through the 1^{st} horn gear polar pair including horn gears 110a, 110e: outer bobbin 120a is a wound wire made of Metal #2 having an outer diameter 0.00175″; inner bobbin 120a′ is a wound wire made of Metal #1 having an outer diameter 0.0012″; inner bobbin 120e′ is a wound wire made of Metal #1 having an outer diameter 0.0012″; and outer bobbin 120e is a wound wire made of Metal #1 having an outer diameter 0.00175″. In the 2^{nd} flexural plane defined through the 2^{nd} horn gear polar pair including horn gears 110b, 110f: outer bobbin 120b is a wound wire made of Metal #1 having an outer diameter 0.0012″; inner bobbin 120b′ is a wound wire made of Metal #2 having an outer diameter 0.00175″; inner bobbin 120f′ is a wound wire made of Metal #1 having an outer diameter 0.00175″; and outer bobbin 120f is a wound wire made of Stainless Steel having an outer diameter 0.0012″. While in the 3^{rd} flexural plane defined through the 3^{rd} horn gear polar pair including horn gears 110c, 110g: outer bobbin 120c is a wound wire made of Stainless Steel having an outer diameter 0.00175″; inner bobbin 120c′ is a wound wire made of Metal #1 having an outer diameter 0.0012″; inner bobbin 120g′ is a wound wire made of Stainless Steel having an outer diameter 0.0012″; and outer bobbin 120g is a wound wire made of Metal #1 having an outer diameter 0.00175″. Along the 4^{th} flexural plane defined through the 4^{th} horn gear polar pair including horn gears 110d, 110h: outer bobbin 120d is a wound wire made of Metal #1 having an outer diameter 0.0012″; inner bobbin 120d′ is a wound wire made of Stainless Steel having an outer diameter 0.00175″; inner bobbin 120h′ is a wound wire made of Metal #2 having an outer diameter 0.00175″; and outer bobbin 120h is a wound wire made of Metal #1 having an outer diameter 0.0012″.

In this example of FIG. 4A, those wires having an outer diameter of 0.0012″ do not cross one another, similarly those wires having an outer diameter of 0.00175″ also do not cross each other. Instead, crossing occurs only with wires differing in outer diameter (e.g., crossing of a wire having an outer diameter of 0.0012″ with a wire having an outer diameter of 0.00175″). As noted in the last two columns on the right in the table of FIG. 7, the aggregate flexural strength (i.e., the radial component (Fz) or the axial component (Fy)) is different among each of the 1^{st} through 4^{th} flexural planes. That is, the higher aggregate flexural strength (i.e., stiffer, more rigid, less flexibility) along the 1^{st} flexural plane in comparison to the lower aggregate flexural strength (i.e., less stiff, less rigid, greater flexibility) along the 2^{nd} flexural plane, which, in turn, is higher in comparison to the aggregate flexural strength along the 4^{th} flexural plane that, in turn, is higher than the aggregate flexural strength along the 3^{rd} flexural plane.

FIG. 4B is a side view of the braided woven construct produced with the arrangement of FIG. 4A creating four distinct spiral patterns extending in the longitudinal/axial direction, wherein each of the four distinct spiral patterns represent those wires associated with each of the 1^{st} through 4^{th} flexural planes (e.g., 1^{st} flexural plane (e.g., coil “A”); 2^{nd} flexural plane (e.g., coil “B”); 3^{rd} flexural plane (e.g., coil “C”); 4^{th} flexural plane (e.g., coil “D”) each having a different aggregate flexural strength.

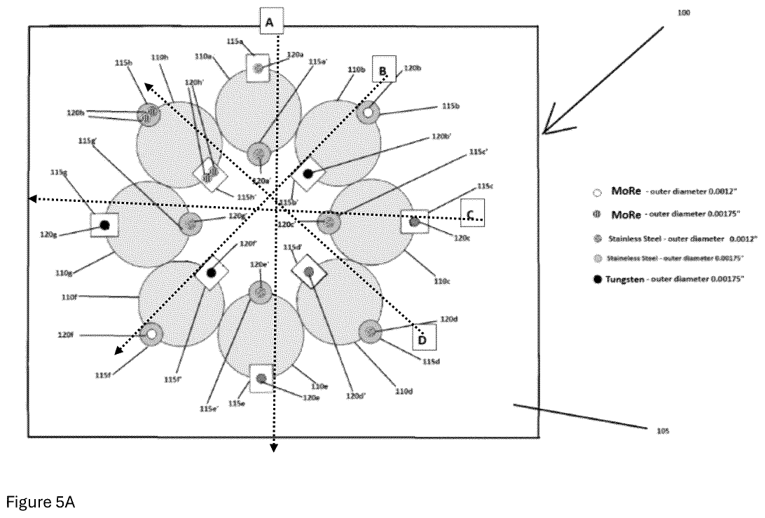

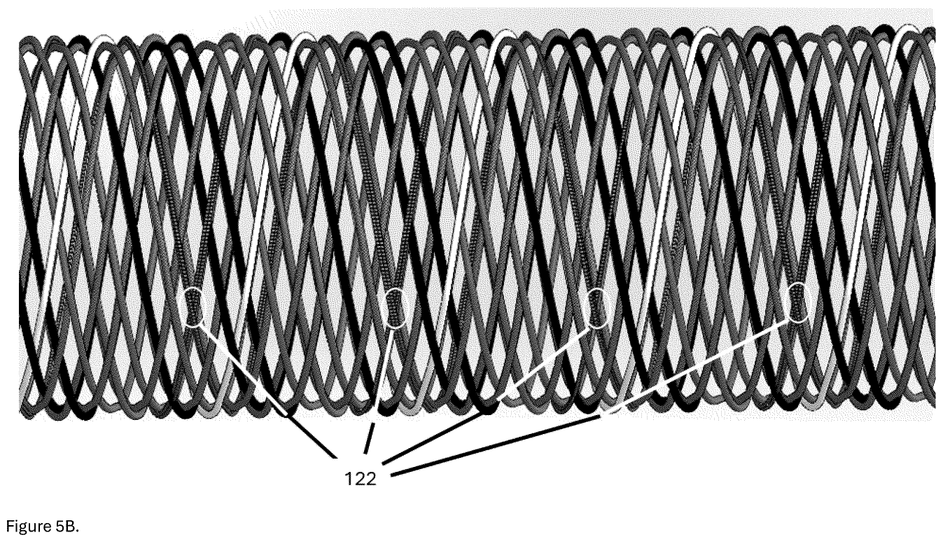

FIG. 5A is a radial cross-sectional view of an example 16 carrier, 8 horn gear braiding system for a fourth example arrangement in which the aggregate flexural strength differs in each of the four flexural planes (e.g., 1^{st} flexural plane defined through the 1^{st} horn gear polar pair; 2^{nd} flexural plane defined through the 2^{nd} horn gear polar pair; 3^{rd} flexural plane defined through the 3^{rd} horn gear polar pair; and 4^{th} flexural plane defined through the 4^{th} horn gear polar pair); wherein horn gear 110h has an inner bobbin carrier and an outer bobbin carrier each carrying two bobbins of a wound wire having identical physical characteristics (e.g., material, outer diameter and radial cross-sectional shape).

The characteristics of the wires for the example arrangement in FIG. 5A are presented in the table of FIG. 7. Specifically, in the 1^{st} flexural plane defined through the 1^{st} horn gear polar pair including horn gears 110a, 110e: outer bobbin 120a is a wound wire made of Stainless Steel having an outer diameter 0.0012″; inner bobbin 120a′ is a wound wire made of Stainless Steel having an outer diameter 0.0012″; inner bobbin 120e′ is a wound wire made of Stainless Steel having an outer diameter 0.0012″; and outer bobbin 120e is a wound wire made of Stainless Steel having an outer diameter 0.00175″. In the 2^{nd} flexural plane defined through the 2^{nd} horn gear polar pair including horn gears 110b, 110f: outer bobbin 120b is a wound wire made of Metal #1 having an outer diameter 0.0012″; inner bobbin 120b′ is a wound wire made of Metal #2 having an outer diameter 0.00175″; inner bobbin 120f′ is a wound wire made of Metal #2 having an outer diameter 0.00175″; and outer bobbin 120f is a wound wire made of Metal #1 having an outer diameter 0.0012″. While in the 3^{rd} flexural plane defined through the 3^{rd} horn gear polar pair including horn gears 110c, 110g: outer bobbin 120c is a wound wire made of Stainless Steel having an outer diameter 0.00175″; inner bobbin 120c′ is a wound wire made of Stainless Steel having an outer diameter 0.0012″; inner bobbin 120g′ is a wound wire made of Stainless Steel having an outer diameter 0.0012″; and outer bobbin 120g is a wound wire made of Metal #2 having an outer diameter 0.00175″. Along the 4^{th} flexural plane defined through the 4^{th} horn gear polar pair including horn gears 110d, 110h: outer bobbin 120d is a wound wire made of Stainless Steel having an outer diameter 0.0012″; inner bobbin 120d′ is a wound wire made of Stainless Steel having an outer diameter 0.00175″; inner bobbin 120h′ is a wound wire made of Metal #1 having an outer diameter 0.00175″; and outer bobbin 120h is a wound wire made of Metal #1 having an outer diameter 0.00175″.

In this example of FIG. 5A, those wires having an outer diameter of 0.0012″ do not cross one another, similarly those wires having an outer diameter of 0.00175″ also do not cross each other. Instead, crossing occurs only with wires differing in outer diameter (e.g., crossing of a wire having an outer diameter of 0.0012″ with a wire having an outer diameter of 0.00175″). As noted in the last two columns on the right in the table of FIG. 7, the aggregate flexural strength (i.e., the radial component (Fz) or the axial component (Fy)) is different among each of the 1^{st} through 4^{th} flexural planes. That is, the aggregate flexural strength along the 4^{th} flexural plane is higher (i.e., stiffer, more rigid, less flexibility) in comparison to the lower aggregate flexural strength (i.e., less stiff, less rigid, greater flexibility) along the 2^{nd} flexural plane, which, in turn, is higher than the aggregate flexural strength along the 3^{rd} flexural plane that, in turn, is higher than the aggregate flexural strength along the 1^{st} flexural plane. In this example of FIG. 5A, two inner bobbins 120h′ and two outer bobbins 120h associated with horn gear 110h represent first and second wire pairs, respectively, wherein those wires in each pair are made of Metal #1 having an outer diameter of 0.00175″.

FIG. 5B is a side view of the braided woven construct produced with the arrangement of FIG. 5A creating four distinct spiral patterns extending in the longitudinal/axial direction, wherein each of the four distinct spiral patterns represent those wires associated with each of the 1^{st} through 4^{th} flexural planes (e.g., 1^{st} flexural plane (e.g., coil “A”); 2^{nd} flexural plane (e.g., coil “B”); 3^{rd} flexural plane (e.g., coil “C”); 4^{th} flexural plane (e.g., coil “D”) each having a different aggregate flexural strength. In FIG. 5B the location of the crossing 122 of the double wires of the two inner bobbins 120h′ and two outer bobbins 120h is illustrated.

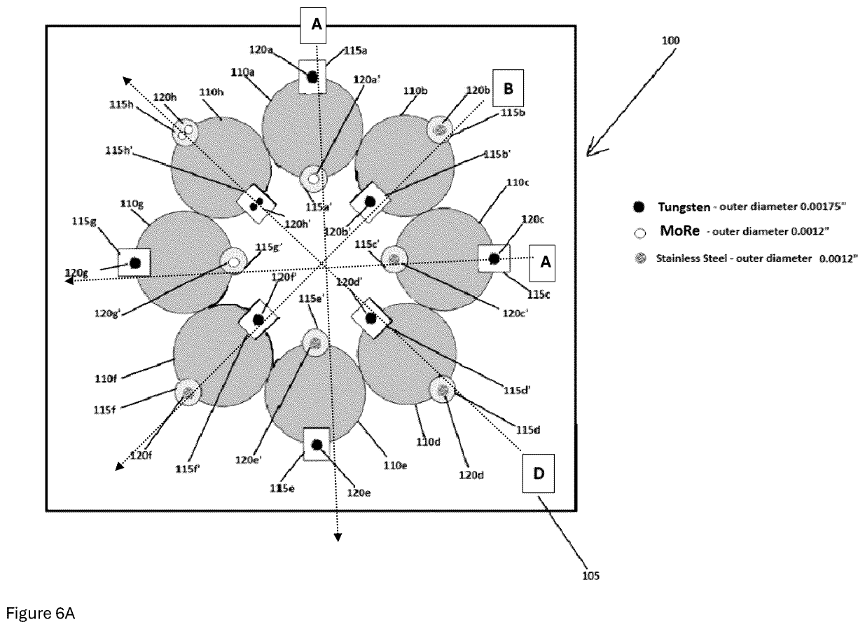

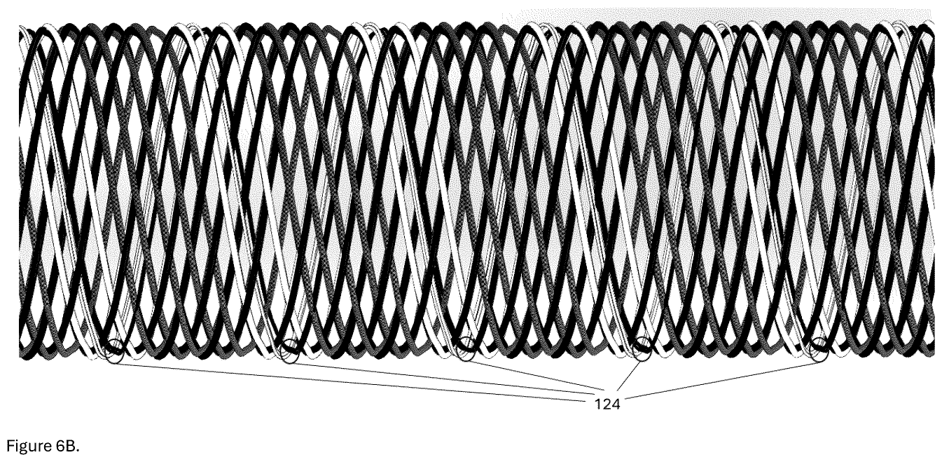

FIG. 6A is a radial cross-sectional view of an example 16 carrier, 8 horn gear braiding system for a fifth example arrangement in which along two flexural planes (e.g., 1^{st} flexural plane defined through the 1^{st} horn gear polar pair; and 3^{rd} flexural plane defined through the 3^{rd} horn gear polar pair) the aggregate flexural strength is equal but larger in comparison to the aggregate flexural strength along the 2^{nd} flexural plane defined through the 2^{nd} horn gear polar pair yet smaller in comparison to the aggregate flexural strength along the 4^{th} flexural plane defined through the 4^{th} horn gear polar pair); wherein horn gear 110h has an inner bobbin carrier and an outer bobbin carrier each carrying two bobbins of a wound wire with the two bobbins of the wound wire fitted on the inner bobbin carrier having different characteristics (e.g., material and outer diameter) from those fitted on the outer bobbin carrier.

While a last illustrative example 8 horn gear, 16 carrier arrangement is shown in FIG. 6A. The characteristics of the wires for the example arrangement in FIG. 6A are presented in the table of FIG. 7. Specifically, in the 1^{st} flexural plane defined through the 1^{st} horn gear polar pair including horn gears 110a, 110e: outer bobbin 120a is a wound wire made of Metal #2 having an outer diameter 0.00175″; inner bobbin 120a′ is a wound wire made of Metal #1 having an outer diameter 0.0012″; inner bobbin 120e′ is a wound wire made of Stainless Steel having an outer diameter 0.0012″; and outer bobbin 120e is a wound wire made of Metal #2 having an outer diameter 0.00175″. In the 2^{nd} flexural plane defined through the 2^{nd} horn gear polar pair including horn gears 110b, 110f: outer bobbin 120b is a wound wire made of Stainless Steel having an outer diameter 0.0012″; inner bobbin 120b′ is a wound wire made of Metal #2 having an outer diameter 0.00175″; inner bobbin 120f′ is a wound wire made of Metal #2 having an outer diameter 0.00175″; and outer bobbin 120f is a wound wire made of Stainless Steel having an outer diameter 0.0012″. While in the 3^{rd} flexural plane defined through the 3^{rd} horn gear polar pair including horn gears 110c, 110g: outer bobbin 120c is a wound wire made of Metal #2 having an outer diameter 0.00175″; inner bobbin 120c′ is a wound wire made of Stainless Steel having an outer diameter 0.0012″; inner bobbin 120g′ is a wound wire made of Metal #1 having an outer diameter 0.0012″; and outer bobbin 120g is a wound wire made of Metal #2 having an outer diameter 0.00175″. Along the 4^{th} flexural plane defined through the 4^{th} horn gear polar pair including horn gears 110d, 110h: outer bobbin 120d is a wound wire made of Stainless Steel having an outer diameter 0.0012″; inner bobbin 120d′ is a wound wire made of Metal #2 having an outer diameter 0.00175″; inner bobbin 120h′ is a wound wire made of Metal #2 having an outer diameter 0.00175″; and outer bobbin 120h is a wound wire made of Metal #1 having an outer diameter 0.0012″.

In this example of FIG. 6A, those wires having an outer diameter of 0.0012″ do not cross one another, similarly those wires having an outer diameter of 0.00175″ also do not cross each other. Instead, crossing occurs only with wires differing in outer diameter (e.g., crossing of a wire having an outer diameter of 0.0012″ with a wire having an outer diameter of 0.00175″). As noted in the last two columns on the right in the table of FIG. 7, the aggregate flexural strength (i.e., the radial component (Fz) or the axial component (Fy)) are equal along the 1^{st} and 3^{rd} flexural planes, but different from each of the 2^{nd} and 4^{th} flexural planes. That is, the aggregate flexural strength along the 4^{th} flexural plane is higher (i.e., stiffer, more rigid, less flexibility) compared to the lower aggregate flexural strength (i.e., less stiff, less rigid, greater flexibility) along the 1^{st} and 3^{rd} flexural planes, which, in turn, is higher than the aggregate flexural strength along the 2^{nd} flexural plane. In this example of FIG. 6A, two inner bobbins 120h′ associated with horn gear 110h represent a first wire pair, each wire of which is made of Metal #2 having an outer diameter of 0.00175″ and two outer bobbins 120h associated with horn gear 110h represent a second wire pair with each wire made of Metal #1 having an outer diameter of 0.0012″). Thus, in this example the two wire pairs differ both in materials and outer diameter. In FIG. 6B, the location of the crossing 124 of the double wires of the two inner bobbins 120h′ and two outer bobbins 120h is illustrated.

FIG. 6B is a longitudinal side view of the produced unitary single piece braided woven construct using the braiding system arrangement of FIG. 2A, wherein the two flexural planes equal in aggregate flexural strength (e.g., 1^{st} flexural plane defined through the 1^{st} horn gear polar pair; and 3^{rd} flexural plane defined through the 3^{rd} horn gear polar pair) create a first spiral pattern extending in the longitudinal direction, while the differential aggregate flexural strength along each of the other two other flexural planes (e.g., 2^{nd} flexural plane defined through the 2^{nd} horn gear polar pair; and 4^{th} flexural plane defined through the 4^{th} horn gear polar pair) create a second and third, respective, spiral pattern extending in the longitudinal direction.

The braided woven construct produced with the arrangement of FIG. 6A creates two distinct spiral patterns extending in the longitudinal/axial direction (e.g., 2^{nd} & 4^{th} flexural planes (e.g., coils “ ”); 2^{nd} flexural plane (e.g., coil “B”); and 4^{th} flexural plane (e.g., coil “D”), associated with each different aggregate flexural strength among the flexural planes.

The example arrangements illustrated and described above are not limiting. Countless other example arrangements are possible for creating a braided woven construct in which the aggregate flexural strength (i.e., stiffness) among the plurality of flexural planes is asymmetrical, non-uniform or differentiated (i.e., not symmetrical, uniform, equal, identical among the plurality of unique flexural planes as defined by the respective horn gear polar pairs). Differentiated aggregate flexural strengths (i.e., differentiated stiffness) among the plurality of flexural planes is realized by: (i) selecting a different characteristic for at least one wire associated with the horn gear polar pair defining an associated flexural plane; and/or (ii) leaving empty at least one bobbin carrier associated with the horn gear polar pair defining an associated flexural plane.

Aspects of the present disclosure are also provided by the following numbered Clauses:

Clause 1 A method for producing a catheter device as a unitary single piece tubular braided woven construct having at least one spiral pattern extending in a longitudinal direction using a multi-carrier braiding system (100) including: a support plate (105) on which is mounted a plurality of horn gears (110a, 110b, 110c, 110d, 110e, 110f, 110g, 110h) collectively arranged radially in a circle defining a collective radial inner surface and a collective radial outer surface; wherein each horn gear of the plurality of horn gears (110a, 110b, 110c, 110d, 110e, 110f, 110g, 110h) is rotatable about a respective central horn gear axis and has associated therewith at least one bobbin carrier (115a, 115a′, 115b, 115b′, 115c, 115c′, 115d, 115d′, 115e, 115e′, 115f, 115f, 115g, 115g′, 115h, 115h′) configured to receive a respective bobbin (120a, 120a′, 120b, 120b′, 120c, 120c′, 120d, 120d′, 120e, 120e′, 120f, 120f′, 120g, 120g′, 120h, 120h′) of a wound wire; and wherein the plurality of horn gears (110a, 110b, 110c, 110d, 110e, 110f, 110g, 110h) represent multiple horn gear polar pairs (110a & 110e; 110b & 110f; 110c & 110g; 110d & 110g) with each horn gear polar pair of the multiple horn gear polar pairs (110a & 110e, 110b & 110f, 110c & 110g; 110d & 110h) representing two horn gears disposed radially in the circle 180 degrees of one another defining a corresponding flexural plane having a corresponding aggregate flexural strength based on the wires associated therewith; the method comprising the step of:
creating the at least one spiral pattern by establishing asymmetric aggregate flexural strengths among the flexural planes.

Clause 2. The method in accordance with clause 1, wherein the step of establishing the asymmetric aggregate flexural strengths among the flexural planes comprises having a differential aggregate flexural strength associated with at least one flexural plane among the flexural planes.

Clause 3. The method in accordance with any of clauses 1-3, wherein the step of causing the differential aggregate flexural strength associated with the at least one flexural plane comprises: (i) selecting at least one different physical characteristic for at least one wire associated with the horn gear polar pair (110a & 110e, 110b & 110f, 110c & 110g; 110d & 110h) defining the at least one flexural plane, wherein the physical characteristic for the at least one wire includes: material, outer diameter and/or radial cross-sectional shape; and/or (ii) leaving empty at least one bobbin carrier associated with the horn gear polar pair (110a & 110e, 110b & 110f, 110c & 110g; 110d & 110h) defining the at least one flexural plane.

Clause 4. The method in accordance with any of clauses 1-3, wherein the differential aggregate flexural strength associated with the at least one flexural plane is unique.

Clause 5. The method in accordance with clause 4, where the differential aggregate flexural strength along each of the flexural planes is unique.

Clause 6. The method in accordance with any of clauses 1-5, wherein the aggregate flexural strength along each flexural plane among the flexural planes represents either an aggregate flexural strength radial component or an aggregate flexural strength axial component.

Clause 7. The method in accordance with any of clauses 1-6, wherein the aggregate flexural strength along one flexural plane among the flexural planes as defined by the corresponding horn gear polar pair (110a & 110e; 110b & 110f; 110c & 110g; 110d & 110h) is at a minimum when an associated bobbin carrier (115a, 115a′, 115b, 115b′, 115c, 115c′, 115d, 115d′, 115e, 115e′, 115f, 115f, 115g, 115g′, 115h, 115h′) is empty.

Clause 8. The method in accordance with any of clauses 1-7, wherein at least one of the bobbin carriers (115a, 115a′, 115b, 115b′, 115c, 115c′, 115d, 115d′, 115e, 115e′, 115f, 115f, 115g, 115g′, 115h, 115h′) is loaded with more than one bobbin (120g, 120h, 120h′).

Clause 9. The method in accordance with any of clauses 1-8, wherein the plurality of horn gears (110a, 110b, 110c, 110d, 110e, 110f, 110g, 110h) is 8, the at least one bobbin carrier (115a, 115a′, 115b, 115b′, 115c, 115c′, 115d, 115d′, 115e, 115e′, 115f, 115f′, 115g, 115g′, 115h, 115h′) associated with each horn gear (110a, 110b, 110c, 110d, 110e, 110f, 110g, 110h) is 2, and the multiple horn gear polar pairs (110a & 110e; 110b & 110f; 110c & 110g; 110d & 110h) is 4.

Clause 10. The method in accordance with any of clauses 1-9, wherein the two bobbin carriers associated with each of the plurality of horn gears (110a, 110b, 110c, 110d, 110e, 110f, 110g, 110h) includes a first bobbin carrier (115a, 115b, 115c, 115d, 115e, 115f, 115g, 115h) disposed on the collective radial outer surface and a second bobbin carrier (115a′, 115b′, 115c′, 115d′, 115e′, 115f, 115g′, 115h′) disposed on the collective radial inner surface.

Clause 11. A catheter device produced using a multi-carrier braiding system (100) including: a support plate (105) on which is mounted a plurality of horn gears (110a, 110b, 110c, 110d, 110e, 110f, 110g, 110h) collectively arranged radially in a circle defining a collective radial inner surface and a collective radial outer surface; wherein each horn gear of the plurality of horn gears (110a, 110b, 110c, 110d, 110e, 110f, 110g, 110h) is rotatable about a respective central horn gear axis and has associated therewith at least one bobbin carrier (115a, 115a′, 115b, 115b′, 115c, 115c′, 115d, 115d′, 115e, 115e′, 115f, 115f′, 115g, 115g′, 115h, 115h′) configured to receive a respective bobbin (120a, 120a′, 120b, 120b′, 120c, 120c′, 120d, 120d′, 120e, 120e′, 120f, 120f′, 120g, 120g′, 120h, 120h′) of a wound wire; and wherein the plurality of horn gears (110a, 110b, 110c, 110d, 110e, 110f, 110g, 110h) represent multiple horn gear polar pairs (110a & 110e; 110b & 110f; 110c & 110g; 110d & 110g) with each horn gear polar pair of the multiple horn gear polar pairs (110a & 110e, 110b & 110f, 110c & 110g; 110d & 110h) including two horn gears disposed radially in the circle 180 degrees of one another defining a corresponding flexural plane having a corresponding aggregate flexural strength based on the wires associated therewith; the device comprising:
a unitary single piece tubular braided woven construct having at least one spiral pattern extending in a longitudinal direction, wherein the at least one spiral pattern is associated with asymmetrical aggregate flexural strengths among the flexural planes.

Clause 12. The device in accordance with clause 11, wherein the asymmetric aggregate flexural strengths among the flexural planes represents a differential aggregate flexural strength associated with at least one flexural plane among the flexural planes.

Clause 13. The device in accordance with any of clauses 11-12, wherein the differential aggregate flexural strength associated with the at least one flexural plane is due to: (i) at least one wire associated with the horn gear polar pair (110a & 110e, 110b & 110f, 110c & 110g; 110d & 110h) defining the at least one flexural plane having at least one different physical characteristic, wherein the physical characteristic for the at least one wire includes: material, outer diameter and/or radial cross-sectional shape; and/or (ii) at least one bobbin carrier associated with the horn gear polar pair defining the at least one flexural plane remaining empty.

Clause 14. The device in accordance with any of clauses 11-13, wherein the differential aggregate flexural strength associated with the at least one flexural plane is unique.

Clause 15. The device in accordance with any of clauses 11-14, where the differential aggregate flexural strength along each of the flexural planes is unique.

Clause 16. The device in accordance with any of clauses 11-15, wherein the aggregate flexural strength along each of the flexural planes represents either an aggregate flexural length radial component or an aggregate flexural strength axial component.

Clause 17. The device in accordance with any of clauses 11-16, wherein the aggregate flexural strength along one of the flexural planes as defined by the corresponding horn gear polar pair (110a & 110e; 110b & 110f; 110c & 110g; 110d & 110h) is at a minimum when an associated bobbin carrier (115a, 115a′, 115b, 115b′, 115c, 115c′, 115d, 115d′, 115e, 115e′, 115f, 115f, 115g, 115g′, 115h, 115h′) is empty.

Clause 18. The device in accordance with any of clauses 11-17, wherein at least one of the bobbin carriers (115a, 115a′, 115b, 115b′, 115c, 115c′, 115d, 115d′, 115e, 115e′, 115f, 115f, 115g, 115g′, 115h, 115h′) is loaded with more than one bobbin (120g, 120h, 120h′).

Clause 19. The device in accordance with any of clauses 11-18, wherein the plurality of horn gears (110a, 110b, 110c, 110d, 110e, 110f, 110g, 110h) is 8, the at least one bobbin carrier (115a, 115a′, 115b, 115b′, 115c, 115c′, 115d, 115d′, 115e, 115e′, 115f, 115f, 115g, 115g′, 115h, 115h′) associated with each horn gear (110a, 110b, 110c, 110d, 110e, 110f, 110g, 110h) is 2, and the multiple horn gear polar pairs (110a & 110e; 110b & 110f; 110c & 110g; 110d & 110h) is 4.

Clause 20. The device in accordance with any of clauses 11-19, wherein the two bobbin carriers associated with each of the plurality of horn gears (110a, 110b, 110c, 110d, 110e, 110f, 110g, 110h) includes a first bobbin carrier (115a, 115b, 115c, 115d, 115e, 115f, 115g, 115h) disposed on the collective radial outer surface and a second bobbin carrier (115a′, 115b′, 115c′, 115d′, 115e′, 115f′, 115g′, 115h′) disposed on the collective radial inner surface.

The descriptions contained herein are examples and not intended in any way to limit the scope of the present disclosure. As described herein, the present disclosure contemplates many variations and modifications of a braiding machine arrangement to create a catheter including a braided woven construct in which the aggregate flexural strengths among the unique flexural planes defined by respective horn gear polar pairs is asymmetrical so that the produced braided woven construct includes at least one spiral pattern of wires of differential aggregate flexural strength extending in the longitudinal direction. Modifications and variations apparent to those having skilled in the pertinent art according to the teachings of this disclosure are intended to be within the scope of the claims which follow.

1. A method for producing a catheter device as a unitary single piece tubular braided woven construct having four distinct spiral patterns extending in a longitudinal direction using a multi-carrier braiding system comprising: a support plate on which is mounted a plurality of horn gears collectively arranged radially in a circle defining a collective radial inner surface and a collective radial outer surface; wherein each horn gear of the plurality of horn gears is rotatable about a respective central horn gear axis and has associated therewith at least one bobbin carrier configured to receive a respective bobbin of a wound wire; and wherein the plurality of horn gears represent multiple horn gear polar pairs with each horn gear polar pair of the multiple horn gear polar pairs representing two horn gears disposed radially in the circle 180 degrees of one another defining a distinct corresponding flexural plane for each of the four distinct spiral patterns having a corresponding aggregate flexural strength based on the wires associated therewith; the method comprising the step of:
creating the four distinct spiral patterns by establishing asymmetric aggregate flexural strengths among the flexural planes; each of the four distinct spiral patterns having a different aggregate flexural strength.

2. (canceled)

3. The method in accordance with claim 1, wherein the step of causing the differential aggregate flexural strength associated with the at least one flexural plane comprises: (i) selecting at least one different physical characteristic for at least one wire associated with the horn gear polar pair defining the at least one flexural plane, wherein the physical characteristic for the at least one wire includes: material, outer diameter and/or radial cross-sectional shape; and/or (ii) leaving empty at least one bobbin carrier associated with the horn gear polar pair defining the at least one flexural plane.

4. (canceled)

5. (canceled)

6. The method in accordance with claim 1, wherein the aggregate flexural strength along each flexural plane among the flexural planes represents either an aggregate flexural strength radial component or an aggregate flexural strength axial component.

7. The method in accordance with claim 1, wherein the aggregate flexural strength along one flexural plane among the flexural planes as defined by the corresponding horn gear polar pair is at a minimum when an associated bobbin carrier is empty.

8. The method in accordance with claim 1, wherein at least one of the bobbin carriers is loaded with more than one bobbin.

9. The method in accordance with claim 1, wherein the plurality of horn gears comprise only eight horn gears, each horn gear being associated with only two bobbin carriers, the eight horn gears being arranged as four polar pairs.

10. The method in accordance with claim 9, wherein the two bobbin carriers associated with each of the plurality of horn gears includes a first bobbin carrier disposed on the collective radial outer surface and a second bobbin carrier disposed on the collective radial inner surface.

11. A catheter device produced using a multi-carrier braiding system comprising: a support plate on which is mounted a plurality of horn gears collectively arranged radially in a circle defining a collective radial inner surface and a collective radial outer surface; wherein each horn gear of the plurality of horn gears is rotatable about a respective central horn gear axis and has associated therewith at least one bobbin carrier configured to receive a respective bobbin of a wound wire; and wherein the plurality of horn gears represent multiple horn gear polar pairs with each horn gear polar pair of the multiple horn gear polar pairs including two horn gears disposed radially in the circle 180 degrees of one another defining a corresponding flexural plane having a corresponding aggregate flexural strength based on the wires associated therewith; the device comprising:
a unitary single piece tubular braided woven construct having four distinct spiral patterns extending in a longitudinal direction, wherein the four distinct spiral patterns are each associated with a distinct asymmetrical aggregate flexural strengths among the flexural planes; each of the four distinct spiral patterns having a different aggregate flexural strength.

12. (canceled)

13. The device in accordance with claim 11, wherein the differential aggregate flexural strength associated with the at least one flexural plane is due to: (i) at least one wire associated with the horn gear polar pair defining the at least one flexural plane having at least one different physical characteristic, wherein the physical characteristic for the at least one wire includes: material, outer diameter and/or radial cross-sectional shape; and/or (ii) at least one bobbin carrier associated with the horn gear polar pair defining the at least one flexural plane remaining empty.

14. (canceled)

15. (canceled)

16. The device in accordance with claim 11, wherein the aggregate flexural strength along each of the flexural planes represents either an aggregate flexural length radial component or an aggregate flexural strength axial component.

17. The device in accordance with claim 11, wherein the aggregate flexural strength along one of the flexural planes as defined by the corresponding horn gear polar pair is at a minimum when an associated bobbin carrier is empty.

18. The device in accordance with claim 11, wherein at least one of the bobbin carriers is loaded with more than one bobbin.

19. The device in accordance with claim 11, wherein the plurality of horn gears comprise only eight horn gears, each horn gear being associated with only two bobbin carriers, the eight horn gears being arranged as four polar pairs.

20. The device in accordance with claim 19, wherein the two bobbin carriers associated with each of the plurality of horn gears includes a first bobbin carrier disposed on the collective radial outer surface and a second bobbin carrier disposed on the collective radial inner surface.
